(19) United States
(12) Patent Application Publication
(10) Pub. No.: US 2016/0355684 A1
(43) Pub. Date: Dec. 8, 2016

(54) ANTHRAQUINONE DYE USED FOR A COLOR FILTER OF A LCD

(71) Applicant: DOW GLOBAL TECHNOLOGIES LLC, Midland, MI (US)

(72) Inventors: Yang Li, Shanghai (CN); Guihong Liao, Shanghai (CN); Matthew S. Remy, Midland, MI (US); Juelin Liu, Shanghai (CN); Hua Ren, Shanghai (CN); Yanping Sun, Shanghai (CN); Chao He, Shanghai (CN); Yu Cai, Shanghai (CN); William J. Kruper, JR., Sanford, MI (US)

(73) Assignee: Dow Global Technologies LLC, Midland, MI (US)

(21) Appl. No.: 15/108,792

(22) PCT Filed: Jan. 29, 2014
(86) PCT No.: PCT/CN14/71769

§ 371 Date: Jun. 29, 2016

(51) Int. Cl. C09B 69/00; C07F 7/08; G02F 1/1335; C07C 221/00

(52) U.S. Cl. CPC C09B 69/008; C07C 221/00; C07F 7/0879; C07F 7/0854; G02F 1/133514; C07C 2103/24

(57) ABSTRACT

An anthraquinone compound which is suitable for forming a color filter used for a liquid crystal display device, a composition containing a resin and the anthraquinone compound, an article having a polymer layer containing the compound and a resin, a color filter containing the compound and a method for synthesis of an asymmetric anthraquinone compound are developed.

### FIELD OF THE INVENTION

The present invention relates to an anthraquinone dye which is suitable for forming a color filter used for a liquid crystal display device, a method for synthesis of an asymmetric anthraquinone compound that is useful in making the anthraquinone dye of one embodiment of this invention, a composition containing an alkaline soluble resin and the anthraquinone dye, an article having a polymer layer comprising the anthraquinone dye and an alkaline soluble resin and a color filter comprising the dye.

### BACKGROUND OF THE INVENTION

Liquid crystal display (LCD) currently dominates the display market because of its excellent performance and small thickness. As a key component of LCD device, translucent color filters play the critical role of generating Red/Green/Blue lights by filtering white light from a back sheet. This capacity originates from the Red/Green/Blue colorants comprised in color filter units. Each colorant possesses a characteristic absorbance spectrum and will show one of the three primary colors when illuminated with white visible light-wavelength ranges from 380 nm to 780 nm. The controlled mixing of primary colors from each color filter unit produced by colorant will generate the final color of pixels. So the efficiency of color filter determines LCD's performance directly.

Normally, the commercialized colorants used in a LCD color filter are exclusively pigments, because they have good stability against heat, light and chemicals. Unfortunately pigments must be ground into micro/nano particles before being added into a color resist to make a color filter due to their intrinsic insolubility property. The agglomerated particle of a colorant causes light scattering. As a result light signal will be lost and transmittance will be low, which means more light energy must be applied to provide enough brightness of the LCD.

In contrast to pigments, dyes are soluble in many materials which ensure that they can be dispersed at molecular level. If dyes are used in a color filter instead of pigments, light scattering will be significantly reduced. So it could be imagined that the dye based color filter will have higher transmittance and energy cost will thus be reduced greatly. However, dye's stability against light, heat and chemical resistance is generally inferior to pigments. As a result, at present, the commercialized LCD color filters are almost exclusively pigments with limited exceptions for a few of pigment-dye hybrid ones.

Some anthraquinone dyes are used for color filters of a LCD. Some anthraquinone dyes have been proposed for color filters, see e.g. U.S. Pat. No. 6,593,483B, U.S. Pat. No. 6,713,641B, U.S. Pat. No. 5,384,377A and US2009/0074820A, but those dyes generally have insufficient thermal stability or insoluble common organic solvent for a color filter.

Although the anthraquinone structure is stable, the low solubility of anthraquinone dyes in an organic solvent prevents the use of anthraquinone dyes for a color filter. Accordingly, an anthraquinone dye which is stable and satisfies the solubility in an organic solvent at the same time is still desired.

1,4-diarylaminoanthraquinone structure shows good thermal stability and solubility in common organic solvent. However, the known method for the synthesis of a 1,4-diarylaminoanthraquinone compound which has differentially substituted aryl groups is quite tedious and complicated, see e.g. U.S. Pat. No. 4,661,293. Therefore, a simple and efficient method for synthesis of 1,4-diarylaminoanthraquinone compound which has different substituents on the aryl groups of the compound is also desired.

### SUMMARY OF THE INVENTION

Inventors of this invention have now found new examples anthraquinone dyes which are stable and have good solubility in an organic solvent and a method for synthesis of an intermediate of the anthraquinone dye.

Therefore, one aspect of this invention relates to an anthraquinone compound which has siloxane structure and is represented by the general formula (1) or formula (2):

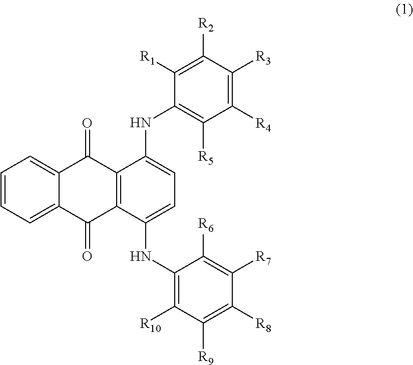

wherein R₁₋₁₀ are independently selected from the group consisting of alkyl group having 1 to 20 carbon atoms, halogen atom, hydrogen atom, hydroxyl group, cyano group, sulfonyl group, sulfo group, sulfato group, aryl group, nitro group, carboxyl group, alkoxy group having 1 to 20 carbon atoms and *—X-L-S1; wherein X is selected from the group consisting of nitrogen atom, oxygen atom, sulfur atom, sulfonyl group, sulfo group and carboxyl group; L is selected from divalent groups consisting of alkylene, oxyalkylene, cycloalkylene, oxygen atom and hetero-containing alkylene; S1 is siloxane containing group represented by —(O—Si(R₁₁)(R₁₂)—)ₙ—O—Si(CH₃)₃, n is an integer from 0 to 100 and R₁₁, R₁₂ are independently selected from the group consisting of hydrogen atom, alkyl group having 1 to 20 carbon atoms and —(O—Si)ₘ—O—Si(CH₃)₃, m is an integer from 0 to 100; * means the position which bonds to the benzene ring of formula (1); and at least one of R₁₋₁₀ is *—X-L-S1:

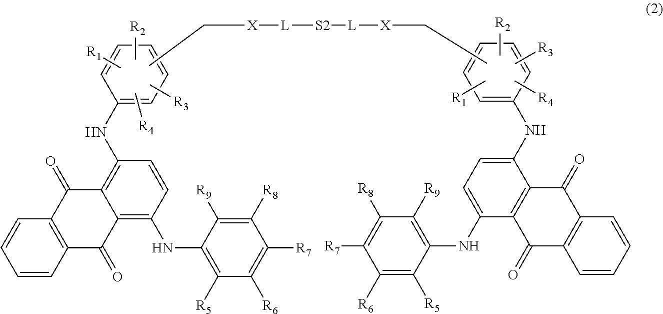

wherein R₁₋₉ are independently selected from the group consisting of alkyl group having 1 to 20 carbon atoms, halogen atom, hydrogen atom, hydroxyl group, cyano group, sulfonyl group, sulfo group, sulfato group, aryl group, nitro group, carboxyl group and alkoxy group having 1 to 20 carbon atoms, X is selected from the group consisting of nitrogen atom, oxygen atom, sulfur atom, sulfonyl group, sulfo group and carboxyl group, L is selected from divalent groups consisting of alkylene, oxyalkylene, cycloalkylene, oxygen atom and hetero-containing alkylene; S2 is siloxane containing divalent group represented by —Si(R₁₀)(R₁₁)—(O—Si(R₁₂)(R₁₃))n-O—Si(R₁₄)(R₁₅)—, R₁₀₋₁₅ are selected from the group consisting of hydrogen atom, alkyl group having 1 to 20 carbon atoms and —(O—Si)ₘ—O—Si(CH₃)₃, m is an integer from 0 to 100, n is an integer from 0 to 100.

Other aspects of this invention relate to; a composition comprising an alkali soluble resin and the anthraquinone compound; an article having a polymer layer formed from the composition disclosed above; and a color filter comprising at least one the anthraquinone compound.

Further aspect of this invention relates to a method for the synthesis of an asymmetric 1,4-diamino anthraquinone compound wherein the amino groups are substituted with different groups selected from alkyl, aryl and alkylaryl groups, comprising the steps of: (A) reacting in the presence of at least one catalyst a mixture of 2,3-dihydro-9,10-dihydroxy-1,4-anthraquinone and 1,4-dihydroxyanthraquinone with a compound represented by the formula (3)

R¹—NH₂ (3)

wherein R¹ is selected from the group consisting of aryl group, alkyl group or an arylalkyl group, R¹ can be substituted by the group selected from the group consisting of hydroxyl group, amino group, thiol group, alkyl group having 1 to 20 carbon atoms, aryl group or combination thereof to form a first intermediate, and

(B) reacting in the presence of at least one catalyst the first intermediate with a compound represented by the formula (4)

R²—NH₂ (4)

wherein R² is selected from the same group of the formula (3), but R¹ and R² are different.

### DETAILED DESCRIPTION OF THE INVENTION

As used throughout this specification, the abbreviations given below have the following meanings, unless the context clearly indicates otherwise: g=gram; mg=milligram; mm=millimeter; min=minute(s); s=second(s); hr.=hour(s); rpm=revolution per minute; ° C.=degree Centigrade. Throughout this specification, “(meth)acrylic” is used to indicate that either “acrylic” or “methacrylic” functionality may be present. As used throughout this specification, the word ‘resin’ and ‘polymer’ is used interchangeably. The word ‘alkaline soluble resin’ and ‘binder’ is used interchangeably.

<Anthraquinone Compound>

The present invention provides an anthraquinone compound represented by the general formula (1) or general formula (2).

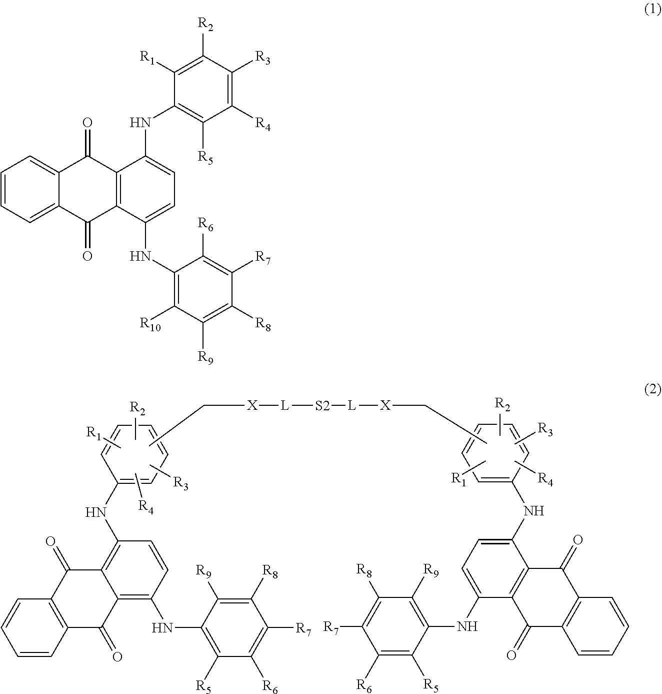

In the formula (1), R₁₋₁₀ are independently selected from the group consisting of alkyl group, halogen atom, hydrogen atom, hydroxyl group, cyano group, sulfonyl group, sulfo group, sulfato group, aryl group, nitro group, carboxyl group, alkoxy group and *—X-L-S1.

The alkyl group has at least 1 carbon atom, and has less than 20 carbon atoms, preferably less than 8 carbon atoms. Examples of the alkyl group are; methyl, ethyl, propyl, butyl, hexyl, octyl, decyl, dodecyl, hexadecyl, octadecyl, isopropyl, sec-propyl, sec-butyl, tert-butyl, 2-ethylhexyl, cyclohexyl, 1-norbornyl and 1-adamantyl.

The alkoxy group has at least 1 carbon atom, and has less than 20 carbon atoms, preferably less than 8 carbon atoms. Examples of the alkoxy group are; methoxyl, ethoxyl, propoxyl, butoxyl, hexoxyl, octoxyl, sec-butoxyl and tert-butoxyl.

In the formula (1), at least one of R₁₋₁₀ are *—X-L-S1. More preferably, at least two of R₁₋₁₀ are *—X-L-S1. The most preferably, at least one of R₁₋₅ and at least one of R₆₋₁₀ are *—X-L-S1. X is selected from the group consisting of nitrogen atom, oxygen atom, sulfur atom, sulfonyl group, sulfo group and carboxyl group. Preferably, X is oxygen atom. L is selected from divalent groups consisting of alkylene, oxyalkylene, cycloalkylene, oxygen atom and hetero-containing alkylene. Alkylene group has at least 1 carbon atom. L is preferably alkylene group. Alkylene group has 10 carbon atoms or less, preferably 5 carbon atoms or less, the most preferably 3 carbon atoms. Si is siloxane containing group represented by —(O—Si(R₁₁)(R₁₂)—)ₙ—O—Si(CH₃)₃. n is an integer from 0 to 100. Preferably n is less than 6, more preferably less than 2. R₁₁ and R₁₂ are independently selected from the group consisting of hydrogen atom, alkyl group having 1 to 20 carbon atoms and —(O—Si)ₘ—O—Si(CH₃)₃. m is an integer from 0 to 100. Preferably m is less than 6, more preferably less than 2. * means the position which bonds to the benzene ring of formula (1).

The anthraquinone compound represented by the formula (1) has preferably 800 or more of molecular weight. High molecular weight of the anthraquinone compound decreases sublimation of the compound during the manufacture of a color film comprising the compound. More preferably, the molecular weight of the anthraquinone compound represented by the formula (1) is 900 or more.

In the formula (2), R₁₋₉ are independently selected from the group consisting of alkyl group, halogen atom, hydrogen atom, hydroxyl group, cyano group, sulfonyl group, sulfo group, sulfato group, aryl group, nitro group, carboxyl group and alkoxy group. The alkyl group has at least 1 carbon atom, and has less than 20 carbon atoms, preferably less than 8 carbon atoms. The alkoxy group has at least 1 carbon atom, and has less than 20 carbon atoms, preferably less than 8 carbon atoms. Examples of the alkyl group and the alkoxy group are same as above.

X is selected from the group consisting of nitrogen atom, oxygen atom, sulfur atom, sulfonyl group, sulfo group and carboxyl group. X is preferably oxygen atom. L is selected from divalent groups consisting of alkylene, oxyalkylene, cycloalkylene, oxygen atom and hetero-containing alkylene. L is preferably alkylene group. Alkylene group has 10 carbon atoms or less, preferably 5 carbon atoms or less, the most preferably 3 carbon atoms. S2 is siloxane containing divalent group represented by —Si(R₁₀)(R₁₁)—(O—Si(R₁₂)(R₁₃))n-O—Si(R₁₄)(R₁₅)—. Both side of S2 are connected to L. R₁₀₋₁₅ are selected from the group consisting of hydrogen atom, alkyl group and —(O—Si)ₘ—O—Si(CH₃)₃. The alkyl group has at least one carbon atom, and has less than 20 carbon atoms. m is an integer from 0 to 100. Preferably m is less than 6, more preferably less than 2. n is an integer from 0 to 100. Preferably n is less than 6, more preferably less than 2.

The anthraquinone dye of the present invention can be used as a mixture. For example, a mixture of anthraquinone dyes which have different substituents as R₁ to R₉ or R₁ to R₁₀ can be used as a mixture. Another example is a compound represented by formula (1) having one *—X-L-S1 group and a compound represented by formula (1) having two *—X-L-S1 groups can be used as a mixture. Of course a compound represented by formula (1) and a compound represented by formula (2) can be used as a mixture. A mixture of two or more of anthraquinone dyes can increase the solubility of dyes in various organic solvents.

The anthraquinone dyes represented by the formula (1) and formula (2) are useful in a color filter of a LCD since those anthraquinone dyes of the invention have excellent thermal stability and high enough solubility for an organic solvent used in the manufacture of LCD such as propylene glycol monomethyl ether acetate (PGMEA).

The anthraquinone compounds of the present invention can be synthesized by the reaction of a siloxane with a corresponding 1,4-bis(arylamine)anthraquinone compounds having unsaturated group. The reaction is known as hydrosilation and Pt, Rh or Ru catalyst is used.

The unsaturated group of the 1,4-bis(arylamine)anthraquinone compounds is incorporated by known reaction such as esterification, etherification, elimination, the Witting reaction or the Friedel-Crafts reaction. One example for the reaction is the reaction of allylbromide with a 1,4-bis(arylamine)anthraquinone compound which has hydroxyl groups on the benzene ring of the compound.

Examples of those reactions are disclosed below.

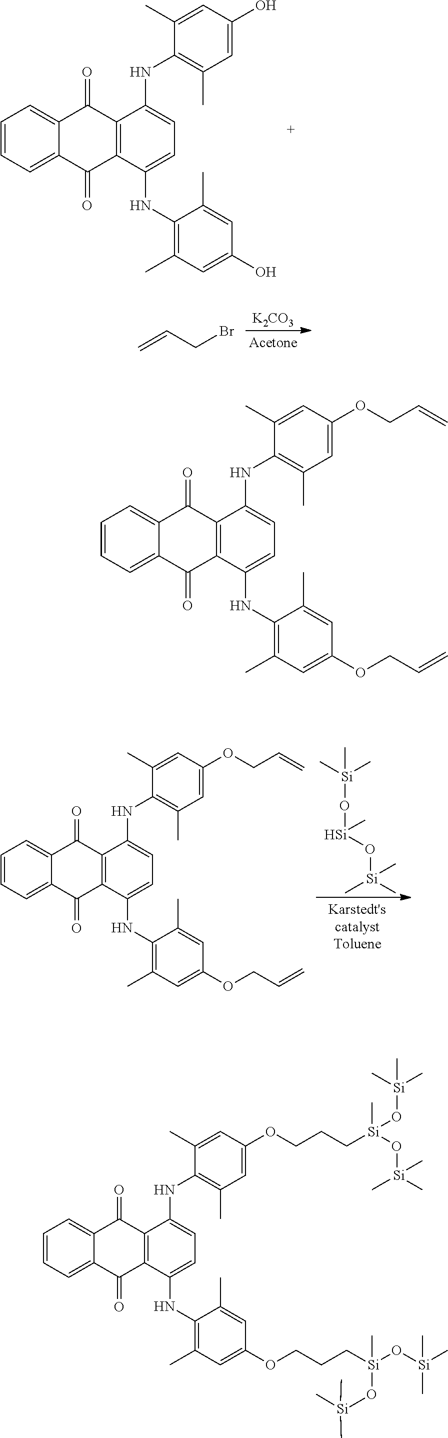

Examples of compounds represented by the formula (1), having various X and L:

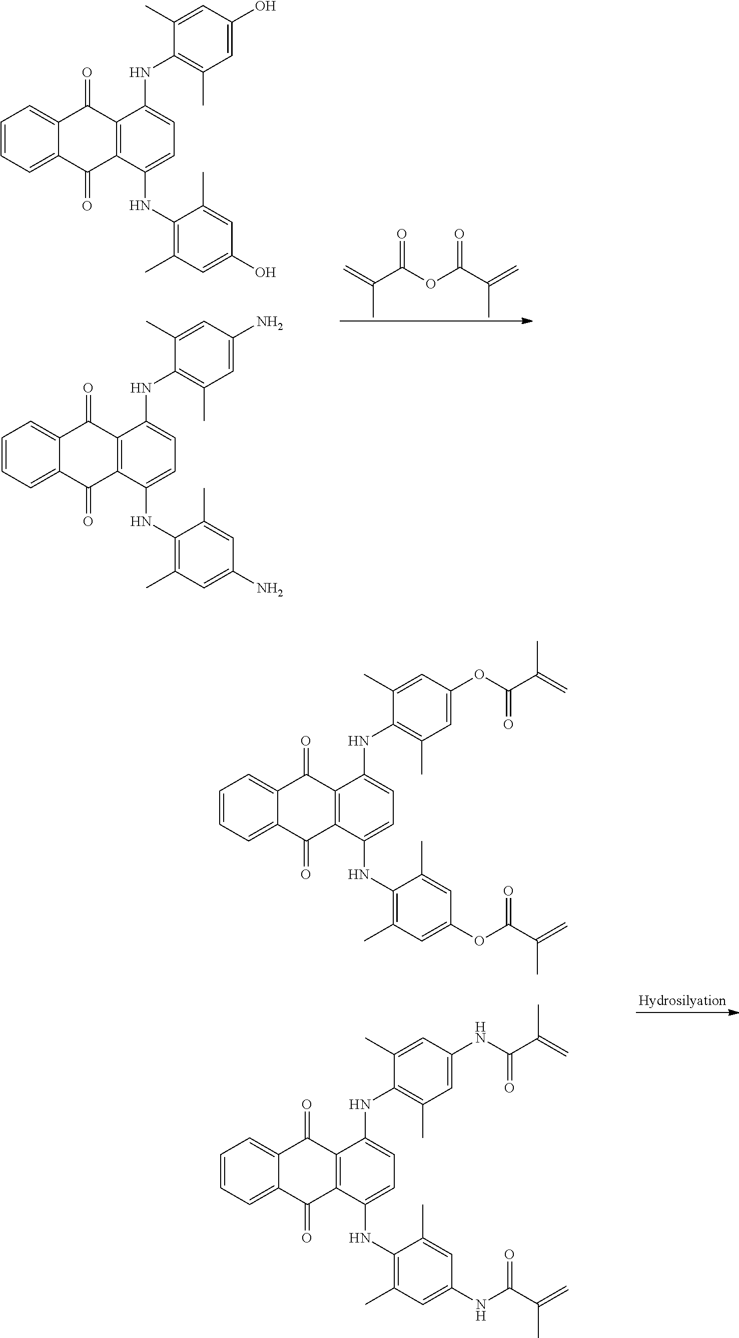

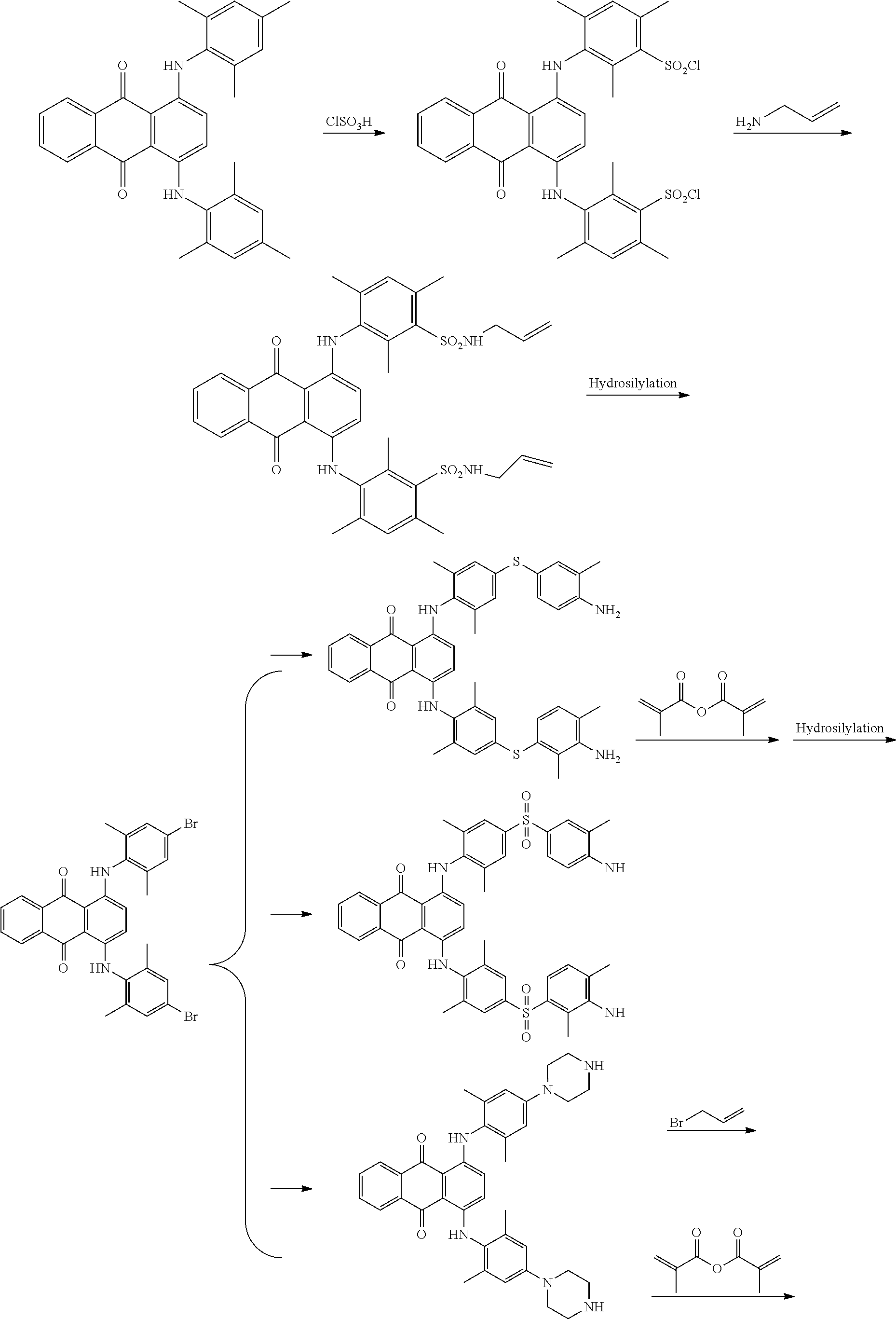

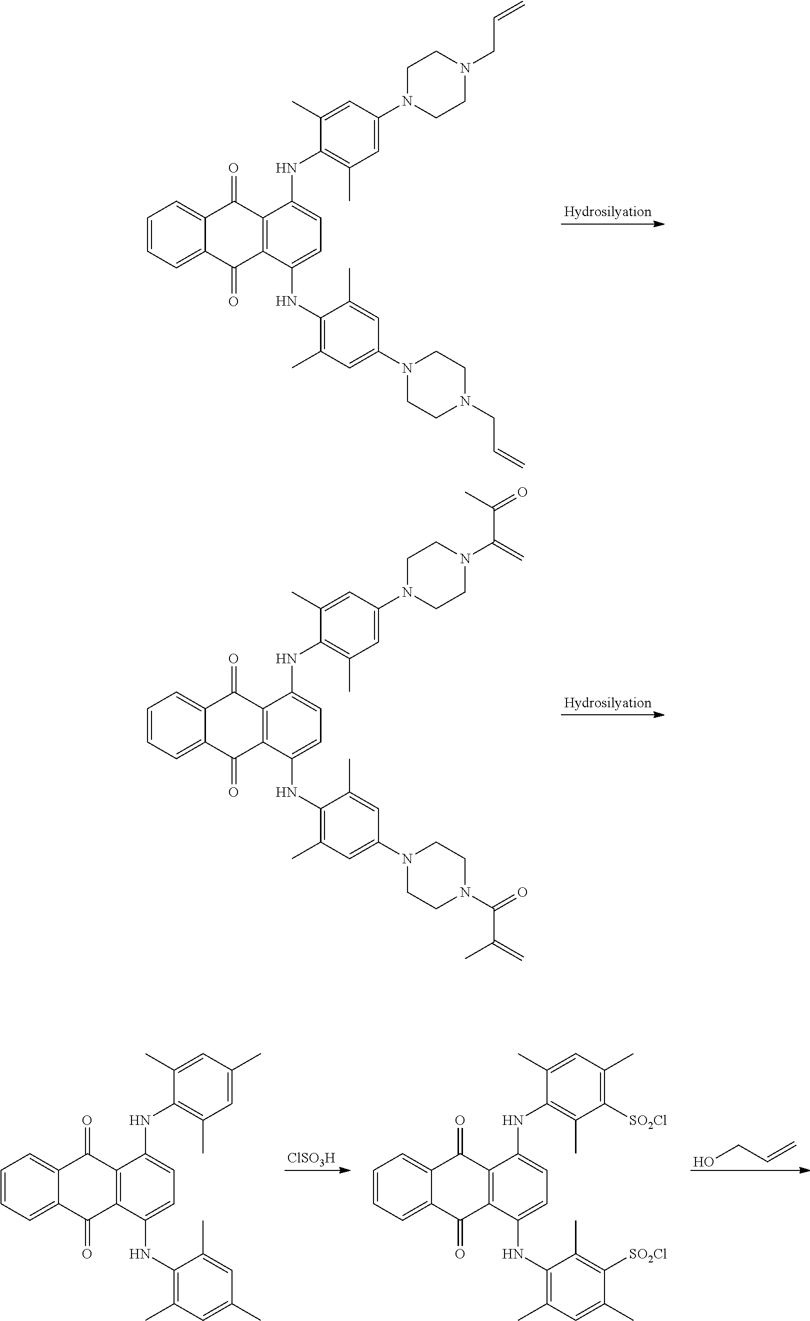

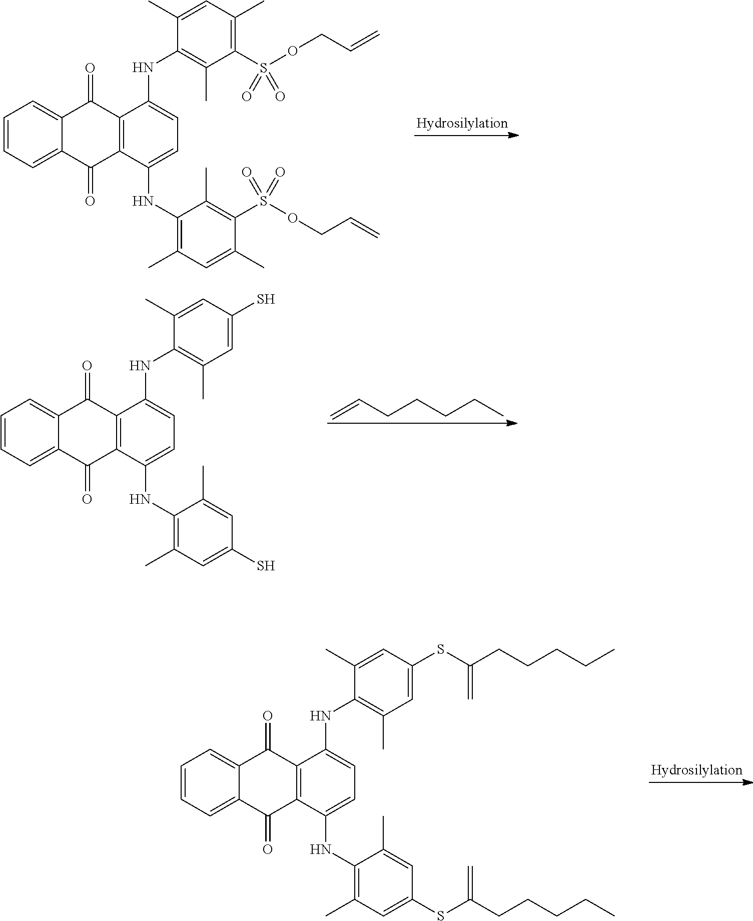

In those reactions, hydrosilylation is conducted same as above.

To synthesize a compound represented by the formula (2), an asymmetric 1,4-bis(arylamine)anthraquinone compound is needed. The “asymmetric” 1,4-bis(arylamine)anthraquinone compound means the two aryl groups (e.g. benzene rings) of the compound have at least one different substituent on the symmetrical positions of the aryl groups. However, the known method for the synthesis of such asymmetric 1,4-bis(arylamine)anthraquinone compound is quite tedious and complicated. Therefore, another subject of the invention is to develop a method for synthesis of an asymmetric 1,4-bis(arylamine)anthraquinone compound which can be used as an intermediate of the compound represented by the formula (2).

The method comprises two steps. The first step is a reaction of a mixture of 2,3-dihydro-9,10-dihydroxy-1,4-anthraquinone and 1,4-dihydroxyanthraquinone with a compound represented by the formula (5) under the presence of at least one catalyst.

Ar¹—NH₂ (5)

In the formula (5), Ar¹ is aryl group which can be substituted by hydroxyl group, amino group, thiol group, alkyl group having 1 to 20 carbon atoms, aryl group or combination thereof.

The reaction is a nucleophilic addition of the amine (formula (5) compound) catalyzed by at least one catalyst to a mixture of 2,3-dihydro-9,10-dihydroxy-1,4-anthraquinone (lecoquinizarin) and 1,4-dihydroxyanthraquinone (quinizarin).

Examples of the catalyst include boric acid and tryalkyl borate, but boric acid is preferable as a catalyst of this reaction.

The mole ratio of 2,3-dihydro-9,10-dihydroxy-1,4-anthraquinone and 1,4-dihydroxyanthraquinone is preferably 1:0.01 to 1:2, more preferably 9:1 to 1:1.5. The mole ratio of the mixture and the compound represented by the formula (5) is, preferably 3:1 to 1:3, more preferably 1.5:1 to 1:1.5. The mole ratio of the mixture and a catalyst is, preferably 4:1 to 1:1, more preferably 3:1 to 1.2:1.

The reaction is conducted in a solvent. Any known solvent can be used unless the solvent does not react with the components used in the reaction. Examples of solvents include n-butanol, N-methylpyrrolidone (NMP), N,N-Dimethylmethanamide (DMF), Tetrahydrofuran (THF), Dimethyl sulfoxide (DMSO), isopropanol, pentanol, dioxane, hexanol and mixture thereof.

The reaction temperature is preferably 80° C. or more, more preferably 100° C. or more. The reaction temperature is preferably 200° C. or less, more preferably 180° C. or less.

The reaction time depends on the reaction temperature and the compound represented by the formula (5), but it is preferably 180 minutes or more, more preferably 300 minutes or more. The reaction time is 24 hours or less, more preferably 18 hours or less.

If lecoquinizarin is used instead of a mixture of lecoquinizarin and quinizarin, lecoquinizarin can react with a compound represented by the formula (5) under the presence of catalyst. However, the yield is lower compared with the reaction using a mixture of lecoquinizarin and quinizarin.

Example of the first step of the method is disclosed below:

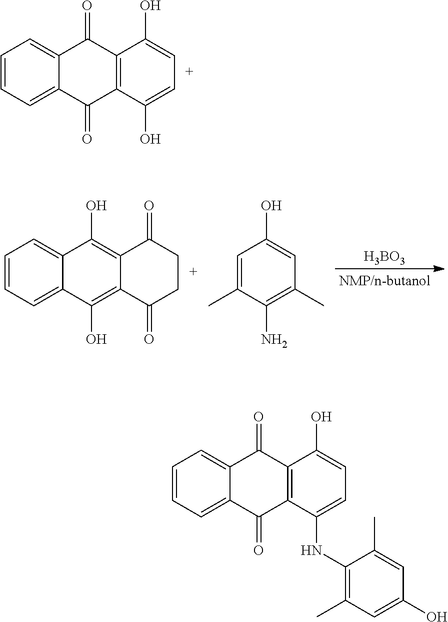

The second step of the method is a reaction of the reaction compound of step 1 with a compound represented by the formula (6) under the presence of at least one catalyst.

Ar²—NH₂ (6)

In the formula (6), Ar² is aryl group which can be substituted by hydroxyl group, amino group, thiol group, alkyl group having 1 to 20 carbon atoms, aryl group or combination thereof. The substituent of the Ar¹ in formula (5) and the substituent of Ar^{e} in the formula (6) are different.

The catalyst used in the second step is preferably boric acid. In addition, zinc and at least one acid are used to help the reaction. Zinc powder or small particle size of metal zinc is used. The size of metal zinc is preferably 10 micrometer or less. Examples of the acid include propionic acid, pivalic acid, trifluoroacetic acid, 2,2-dimethylbutyric acid and mixtures thereof. Preferably, the acid is selected from propionic acid and pivalic acid. More preferably, the acid is pivalic acid because the sterically crowded group of the pivalic acid prevents amide formation (by-product) of the reaction.

Metal zinc and an acid are used to help the reduction of quinone to phenol. Metal zinc is a strong reducer and an acid provides a proton.

The mole ratio of the reaction compound of the first step and the compound represented by the formula (6) is preferably 1:20 to 1:1, more preferably 1:15 to 1:2. The mole ratio of the compound represented by the formula (6) and a catalyst such as boric acid is, preferably 20:1 to 1:1, more preferably 10:1 to 2:1.

The reaction temperature is preferably 80° C. or more, more preferably 100° C. or more. The reaction temperature is preferably 200° C. or less, more preferably 180° C. or less.

The reaction time depends on the reaction temperature and the compound represented by the formula (6), but it is preferably 1 hour or more, more preferably 4 hours or more, the most preferably 5 hours or more. The reaction time is 24 hours or less, more preferably 18 hours or less.

The reaction compound of the first step can be purified using column chromatography or any other known methods between the first step and the second step.

Example of the second step of the method is disclosed below:

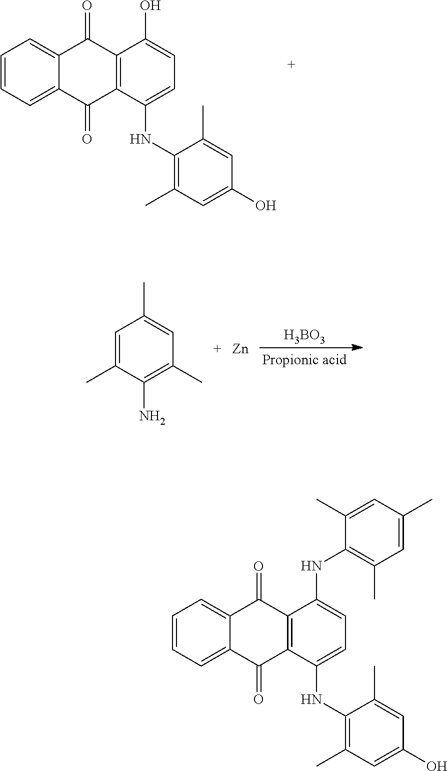

The compound represented by the formula (2) can be synthesized from the reaction compound of the step 2 and a compound having unsaturated group same as the method for the compound represented by the formula (1). Example of such reaction is disclosed below:

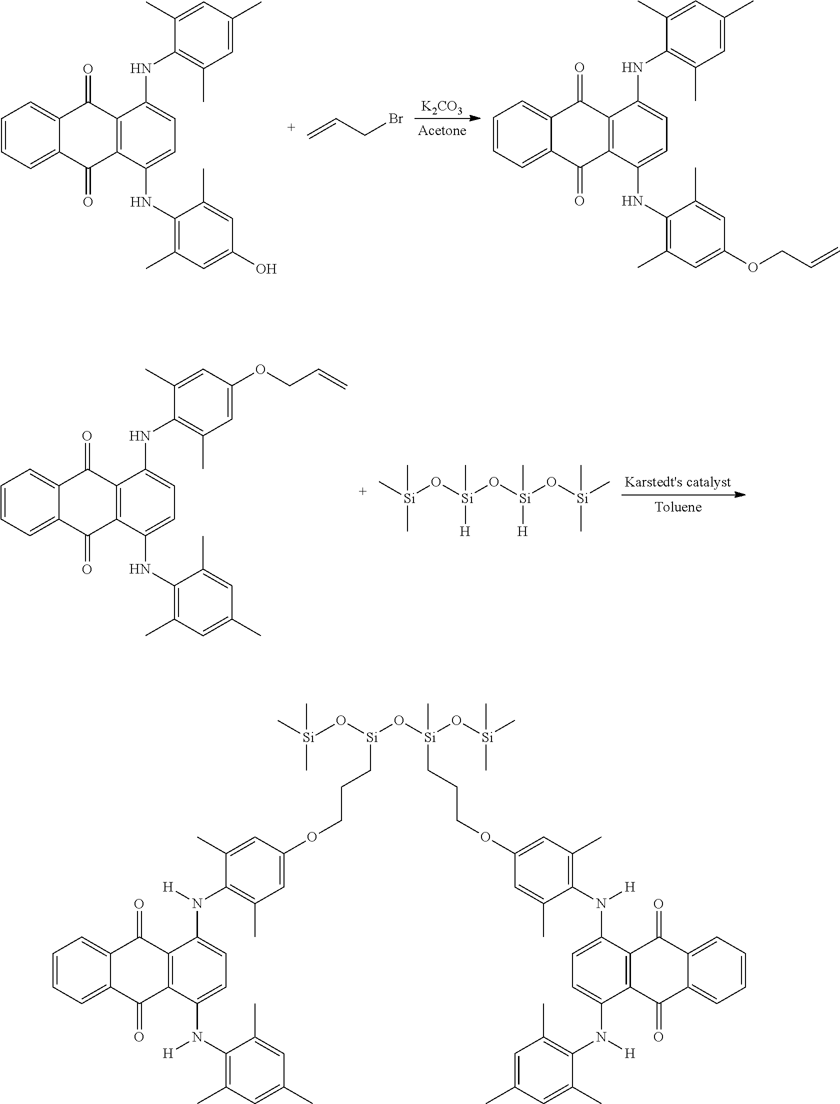

<Composition>

The composition of the present invention comprises the compound as recited at least one of formula (1) and formula (2) and a resin. The resin is preferably alkaline soluble resin. The composition preferably additionally comprises a cross-linker (cross-linking agent), a solvent and a radiation-sensitive compound such as a photo initiator. The composition can form a film useful for a color filter.

The content of the dye as recited in formula (1) or formula (2) in the composition of the present invention varies depending on each molar absorption coefficient and required spectral characteristics, film thickness, or the like, but it is preferably at least 1 wt %, more preferably at least 2 wt %, the most preferably at least 5 wt % based on the total solid contents of the composition. The preferable content is less than 55 wt %, more preferably less than 45 wt %, most preferably less than 35 wt % based on the total solid contents of the composition.

The composition of the present invention can comprises other coloring materials in addition to the dye as recited in formula (1) and formula (2). Normally the use of additional coloring material is determined from the required spectral characteristics of a material to be formed from the composition.

The alkaline soluble resin is also known as ‘binder’ in this technical art. Preferably, the alkaline soluble resin is dissolved in an organic solvent. The alkaline soluble resin can be developed with an alkaline solution such as tetramethyl ammonium hydroxide aqueous solution (TMAH) after forming a film.

The alkaline soluble resin (binder) is normally a linear organic polymer. The binder optionally has a crosslinkable group within the polymer structure. When the composition of the present invention is used as a negative type photosensitive composition, such crosslinkable group can react and form crosslink by exposure or heating so that the binder becomes a polymer which is insoluble in alkaline.

Many kinds of binder are known in this art. Examples of such binder are; (meth)acrylic resin, acrylamide resin, styrenic resin, epoxy resin, polysiloxane resin, phenolic resin, novolak resin, and co-polymer or mixture of those resins. In this application, (meth)acrylic resin (polymer) includes copolymer of (meth)acrylic acid or ester thereof and one or more of other polymerizable monomers. For example, acrylic resin can be polymerized from acrylic acid and/or acrylic ester and any other polymerizable monomers such as styrene, substituted styrene, maleic acid or glycidyl (meth)acrylate.

The binder preferably has at least 1,000 of weight-average molecular weight (Mw), more preferably at least 2,000 of Mw measured by a GPC method using polystyrene as a standard. At the same time, the binder preferably has less than 200,000 of Mw, more preferably less than 100,000 of Mw measured by the same method described above.

The amount of the binder used in the composition of the present invention is preferably at least 10 wt %, more preferably at least 20 wt % based on the total solid contents of the composition. At the same time, the preferable amount of the binder is less than 80 wt %, more preferably less than 50 wt %, the most preferably less than 30 wt % based on the total solid contents of the composition.

The composition of this invention optionally further comprises a cross-linking agent to obtain a further hardened material. It is also known as a radical-polymerizable monomer. When the composition of this invention is used as a negative type photosensitive composition, such cross-linking agent can form a crosslink by exposure or heating and contribute to get a further hardened material. Well known cross-linking agent can be used for the composition of this invention. Examples of cross-linking agents are epoxy resin such as bisphenol A diglycidyl ether, ethyleneglycol diglycidyl ether, butanediol diglycidyl ether, diphentaerythritol pentaglycidyl ether or dipentaerythritol hexaglycidyl ether and substituted nitrogen containing compound such as melamine, urea, guanamine or glycol uril.

The composition of this invention optionally further comprises a solvent. The solvent to be used for the composition is not limited, but preferably selected from the solubility of components of the composition such as binder or anthraquinone dye. Examples of the preferable solvent include esters such as ethylacetate, n-butyl acetate, amyl formate, butyl propionate or 3-ethoxypropionate, ethers such as diethylene glycol dimethyl ether, ethylene glycol monomethyl ether or propylene glycol ethyl ether acetate and ketones such as methylethylketone, cyclohexanone or 2-heptanone.

When the composition of this invention is a negative type radiation-sensitive composition, the composition preferably comprises a photo initiator. Photo initiator also called as photopolymerization initiator and including radical initiator, cationic initiator and anionic initiator. Examples of a photo initiator include; oxime ester type initiator, sulfonium salts initiator, iodide salts initiator and sulfonate initiator.

The composition of this invention can comprise other radiation-sensitive compound such as a radiation sensitive resin or a photo acid generator.

<Polymer Layer>

The composition of the present invention described above can form a polymer layer on an article. The polymer layer also described as ‘polymer film’ in the specification.

The contents of at least one anthraquinone compounds as recited in formula (1) and formula (2) in the polymer layer is depend on the required color of the film, but at least 1 wt %, preferably at least 5 wt % based on the polymer layer. At the same time, the content of the compounds is less than 50 wt %, preferably less than 35 wt % based on the polymer layer. The polymer layer also comprises an alkaline soluble resin which is disclosed above.

The polymer layer optionally comprises a photo initiator, a photo acid generator, a radiation sensitive resin and a crosslink agent disclosed above.

The method of forming the polymer layer on an article comprises the steps of; mixing the anthraquinone compound of this invention with a resin and solvent, coating the mixture on an article which supports a layer and heating the article to form a polymer layer (film). Optionally, the method comprises one or more of steps of exposing a layer (film) or curing a layer to form crosslinked stable layer.

The resin and the solvent used to the method for forming the polymer layer are same as the one disclosed above.

Examples of an article which supports a layer (film) are glass, metal, silicon substrate and metal oxide coated material.

Any coating method can be used for the coating step, such as rotation coating, cast coating or roll coating.

The thickness of the layer (film) varies depending on the required properties of the film. The thickness of the layer is 0.1 to 5 micron, preferably 0.5 to 3 micron.

The layer (film) has high transmittance and thermal stability from the properties of the anthraquinone dye of this invention. The anthraquinone dye can be dissolved in an organic solvent, and has high thermal stability. Therefore the dye does not prevent the transmittance of a film and does not decrease the thermal stability of the film. Such property is important for a color filter of LCD. Therefore, the layer (film) of the present invention is useful as a color filter of LCD.

<Color Filter>

The color filer of this invention comprises at least one anthraquinone compound as recited in formula (1) and formula (2). The layer (film) disclosed above can be used for the color filter. Normally, a color filter has multiple units which made from colored films comprising Red/Green/Blue colorants.

The contents of the anthraquinone compound of this invention in a colored film for a color filter is same as the film disclosed above, at least 1 wt %, more preferably at least 5 wt % based on the total weight of the colored film. At the same time, the content is less than 50 wt %, preferably less than 35 wt % based on the total weight of the colored film.

A film used for a color filter can be formed by the following steps; coating a solution comprising at least one compound as recited in formula (1) and formula (2), binder, a photo initiator and solvent to form a radiation sensitive composition layer on a material, exposing the layer through a patterned mask, and developing the layer with an alkaline solution. Moreover, a curing step of further heating and/or exposing the layer after developing step may be conducted as needed.

Since a color filter comprises three colored films which comprise R/G/B colorant, the steps of forming each colored film are repeated, then a color filter having such three colored films are obtained.

Other Applications for the Method of this Invention

While the method of this invention is used for the synthesis of an intermediate of the compound represented by the formula (2), the method can apply synthesis of other 1,4-diamino anthraquinone compounds wherein the amino groups of the anthraquinone compounds are substituted by different groups selected from the group consisting of alkyl group, aryl group and alkylaryl group. Examples of those reactions are disclosed below.

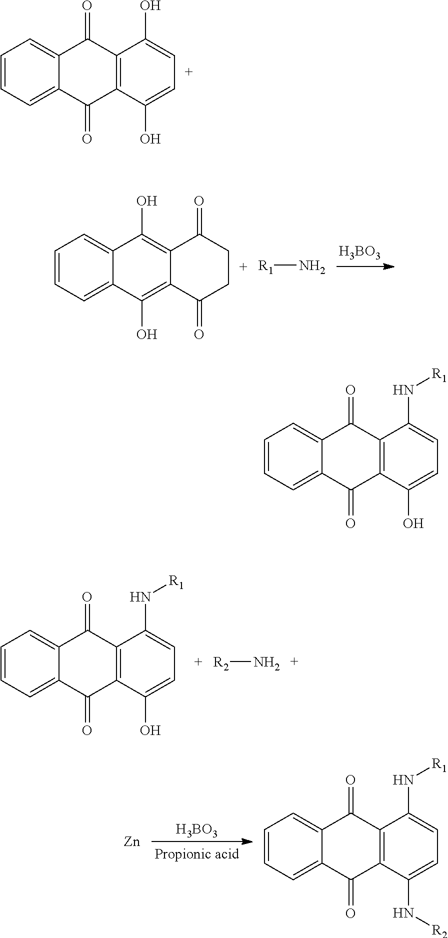

R₁ and R₂ are independently selected from alkyl group having 1 to 20 carbon atoms, aryl group and alkyl aryl group having 1 to 20 carbon atoms. R₁ and R₂ may be substituted by hydroxyl, amino and thiol groups, at least one carbon atom of R₁ and R₂ may be replaced by hetero atoms, and R₁ and R₂ are different.

Another example of the reaction is disclosed below.

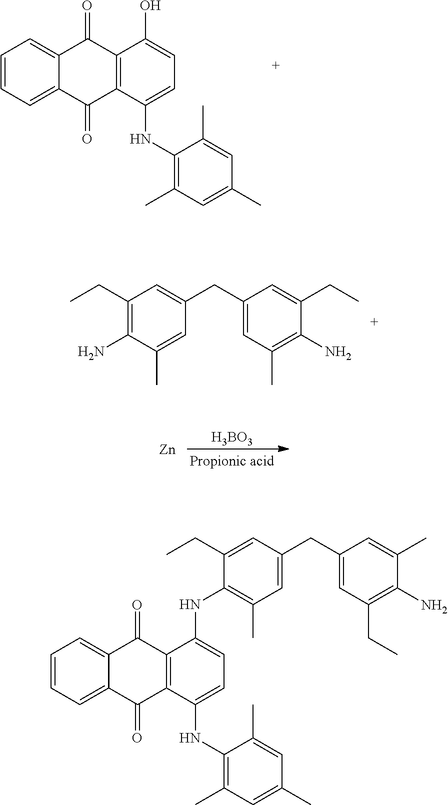

¹H NMR (CDCl₃, ppm): 11.83 (s, 1H), 11.78 (s, 1H), 8.41 (m, 2H), 7.74 (m, 2H), 6.98 (s, 1H), 6.92 (s, 3H), 6.79 (d, 2H), 6.54 (s, 2H), 3.81 (s, 2H), 3.52 (s, 2H), 2.52 (m, 4H), 2.29 (s, 3H), 2.14 (m, 12H), 1.10 (t, 6H). ESI-MS (m/z, Ion, Formula): 622.3458, (M+H)⁺, C₄₂H₄₄N₃O₂, (theoretical mass 621.34)

### EXAMPLES

### Example 1

### Synthesis of an Intermediate

A intermediate of a compound represented by the formula (2) is synthesized following the formulas below.

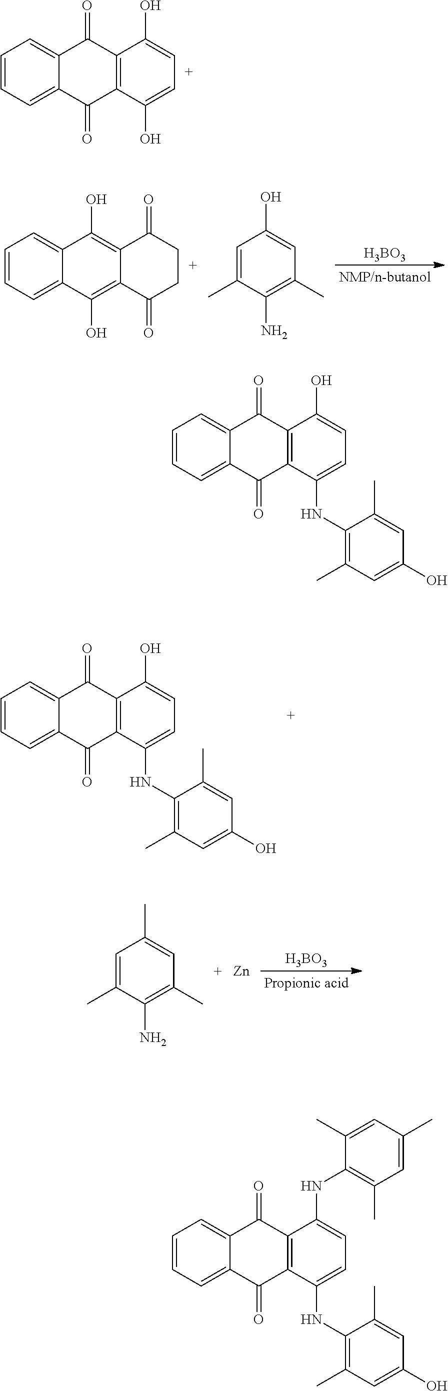

A mixture of 0.44 g (1.82 mmol) of 2,3-dihydro-9,10-dihydroxy-1,4-anthracenedione (10% quinizarin as impurity), 0.44 g (1.82 mmol) quinizarin, 0.15 g (2.44 mmol) of boric acid, 5 mL n-butanol was refluxed under N₂ for 10 min. Then, 0.37 g (2.73 mmol) of 2,6-dimethyl-4-hydroxylaniline was dissolved into 2 mL NMP and added dropwise during 2 hours. The reaction mixture was stirred at reflux under N₂ overnight. After cooling to room temperature, n-butanol was distilled off by vacuum evaporator. Water was added to take NMP, solid was washed with water and dried. After dissolved into THF and mixed with silica gel, the product with purple color was obtained by column chromatography with the yield of 64%. 1H NMR (CDCl₃, ppm): 13.70 (s, 1H), 11.21 (s, 1H), 8.38 (m, 2H), 7.80 (m, 2H), 7.12 (d, 1H), 6.73 (d, 1H), 6.65 (s, 2H), 4.81 (s, 1H), 2.14 (s, 6H). ESI-MS (m/z, Ion, Formula): 360.123, (M+H)⁺, C₂₂H₁₈NO₄, (theoretical mass 359.12).

A mixture of 1.00 g of 1-((4-hydroxy-2,6-dimethylphenyl)amino)-4-hydroxyl-anthraquinone, 3.76 g of trimethylaniline, 0.20 g of boric acid, 0.20 g of zinc dust, and 2.00 g propionic acid was heated at 160° C. for 6 hours in an oil bath under N₂. The reaction mixture was poured into 100 mL of crushed ice containing 10 mL of concentrated hydrochloric acid. The residue remaining in the reaction flask was transferred to the ice-acid mixture using 8 mL of propionic acid. The stirred mixture was then filtered to give the mixed product. Then washed with 5% hydrochloric acid, and water. After dried, the final product was purified by silica gel column using methylene chloride and methanol as eluent. The asymmetrically substituted 1,4-diarylamino-anthraquinone derivative with blue color was obtained with the yield of 81%. ¹H NMR (CDCl₃, ppm): 11.79 (s, 1H), 11.68 (s, 1H), 8.42 (m, 2H), 7.75 (m, 2H), 6.94 (s, 2H), 6.61 (s, 2H), 6.57 (s, 2H), 5.30 (s, 1H), 2.30 (s, 3H), 2.13 (s, 12H). ESI-MS (m/z, Ion, Formula): 477.2179, (M+H)⁺, C₃₁H₂₉N₂O₃, (theoretical mass 476.21).

### Example 2

### Preparing an Anthraquinone Dye and a Film Comprising the Dye

An anthraquinone dye (Dye 1) disclosed below was used in Example 2.

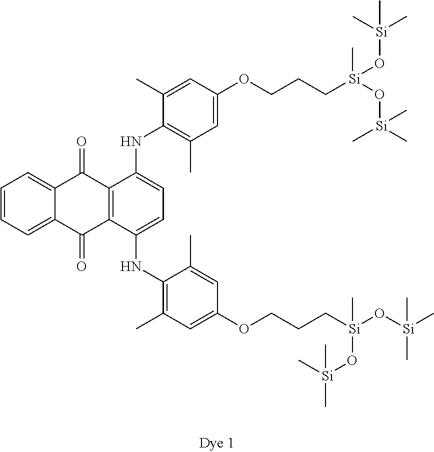 [SMILES: Cc1cc(OCCC[Si](C)(O[Si](C)(C)C)O[Si](C)(C)C)cc(C)c1Nc1ccc(Nc2c(C)cc(OCCC[Si](C)(O[Si](C)(C)C)O[Si](C)(C)C)cc2C)c2c1C(=O)c1ccccc1C2=O]

Synthesis of Dye 1

a. Preparation of Bis-Allyl Modified Anthraquinone (DiAA)

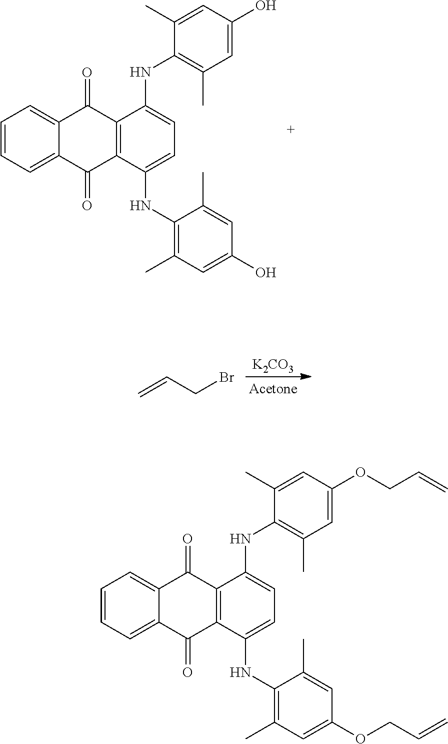

A mixture of 1,4-bis(2′,6′-dimethyl-4′-hydroxyanilino)anthraquinone (2.00 g, 4.15 mmol), allylbromide (1.25 g) and anhydrous K₂CO₃ (1.75 g) in dry acetone (30 mL) was refluxed under N₂ overnight. The reaction mixture then cooled to room temperature, filtered through sintered funnel to remove solid residue, and the filtrate was evaporated to the dryness. The residue was purified by column chromatography. The blue colorant (DiAA) was obtained with the yield of 92%. ¹H NMR (CDCl₃, ppm): 11.71 (s, 2H), 8.42 (m, 2H), 7.55 (m, 2H), 6.69 (m, 4H), 6.57 (m, 2H), 6.05 (m, 2H), 5.36 (m, 2H), 4.52 (d, 4H), 2.16 (s, 12H). ESI-MS (m/z, Ion, Formula): 559.2594, (M+H)⁺, C₃₆H₃₅N₂O₄, (theoretical mass 558.25).

b. Preparation of Bis-Siloxane Modified Anthraquinone (DiSA)

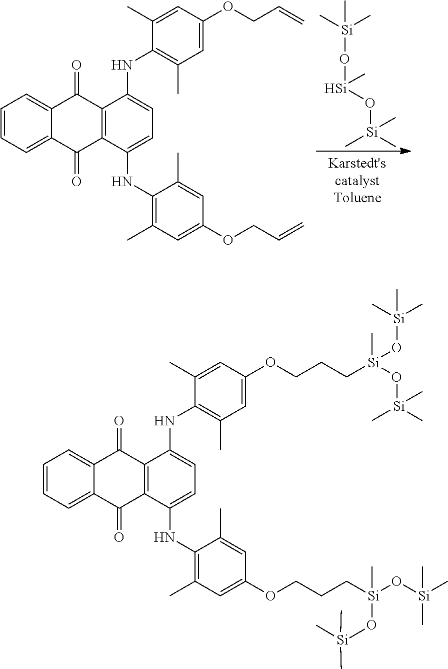

DiAA (1.00 g, 1.79 mmol) was dissolved in anhydrous toluene (20 mL) under N₂. 1,1,1,3,3,5,5,5-Heptamethyltrisiloxane (1.00 g, 4.47 mmol) was injected through a septum, followed by the addition of a drop of Karstedt's catalyst (platinum divinyltetramethy-siloxanecomplex in xylene, 3 wt). The resulting mixture was stirred at 50° C. overnight. The solution was evaporated under reduced pressure. The crude product was purified by chromatography on silica. Isolated yield=79% (DiSA). The weight loss of Dye1 at 230° C. for 1 h in air from TGA is 7.74%. Absorption spectra: maximum peaks at 582 nm (log E=4.29) and 626 nm (log E=4.33). ¹H NMR (CDCl₃, ppm): 11.62 (s, 2H), 8.32 (m, 2H), 7.65 (m, 2H), 6.56 (m, 4H), 6.47 (m, 2H), 3.78 (t, 4H), 2.04 (s, 12H), 1.70 (m, 4H), 0.48 (t, 4H), 0.02 (bs, 36H), −0.05 (s, 6H). ESI-MS (m/z, Ion, Formula): 1003.4465, (M+H)⁺, C₅₀H₇₉N₂O₈Si₆, (theoretical mass 1002.44).

c. Preparation of a Color Resist and a Color Film Comprising an Anthraquinone Dye

0.15 g of Dye 1, 1.35 g PGMEA and 1.00 g of alkaline soluble acrylic resin solution (MIPHOTO RPR4022, supplied from Miwan Commercial Co., Ltd., 25 to 35% of solid content in (methyl 3-methoxypropionate)) were mixed and stirred for overnight at room temperature using a shaker. The solution was filtered with a 0.45 μm Acrodisc CR PTFE filter to get rid of big particles. Then the filtered solution was spin coated onto a clean glass substrate with 400 rpm spin speed for 18 seconds. The obtained film was first dried at 90° C. under air atmosphere for 30 minutes, and then hard baked at 230° C. under air atmosphere for 1 hour. The CIE values (xyY values and lab values) and the UV-Vis were measured before and after the hard bake.

Film thickness, transmittance and chromaticity coordinates of the obtained film were measured as disclosed below. Film thickness of the film was about 1.0 micron. Chromaticity coordinates measured by UltraScan Pro (Hunterlab) colorimeter was, x=0.15, y=0.18 and Y=18.30.

The obtained dry film was further baked at 230° C. under air for 1 hour to evaluate thermal stability of the film. Optical performance after baking (ΔE_{ab} value) was 1.8, and the one of after further baking was 2.7. A smaller ΔE_{ab} value indicates better heat resistance. The result is shown in Table 1.

<Performance Evaluation>

### (1) Thermal Stability of Dyes (Mass Loss Measured by TGA):

The thermal stability of dye itself was determined by the mass loss of dye measured by TGA under air atmosphere at 230° C. for 1 hour. This evaluation reflects chemical stability of the dye itself.

### (2) Film Thickness:

Film thickness is measured by scanning the difference in height across the boundary of film and glass substrate with atomic force microscope.

### (3) Chromaticity Coordinates:

The chromaticity coordinate of film on a glass sheet is directly recorded with UltraScan Pro (Hunterlab) colorimeter. The light source is D65/10.

### (4) Thermal Stability of Films (Chromaticity):

The wet film after spin coating is dried in oven at 90° C. for 30 minutes and then soft baked at 150° C. for 15 minutes. The chromaticity coordinates (L, a, b) are recorded with UltraScan Pro (Hunterlab) colorimeter. D65/10 light source is used and results are based on CIE Lab coordinates. After that the film is hard baked at target temperature (230° C.) for 1 hour and the new chromaticity coordinates (L′, a′, b′) are recorded with the method above. The thermal stability of a film is indicated by the difference of chromaticity coordinate after baking at 230° C. represented by the following formula;

ΔE=\sqrt{(L−L′)²+(a−a′)²+(b−b′)²}

### Examples 3 and 4

Two anthraquinone dyes (Dye2 and Dye3) disclosed below were used in Inventive Examples 2 and 3.

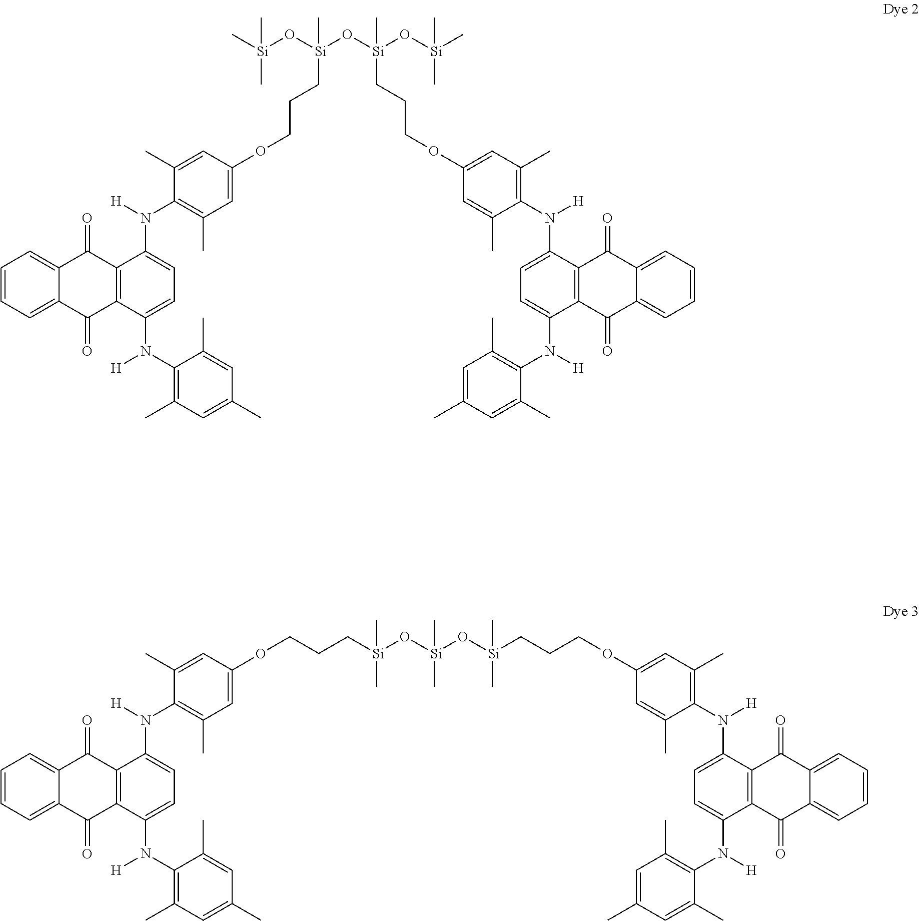 [SMILES: Cc1cc(C)c(Nc2ccc(Nc3c(C)cc(OCCC[Si](C)(O[Si](C)(C)C)O[Si](C)(CCCOc4cc(C)c(Nc5ccc(Nc6c(C)cc(C)cc6C)c6c5C(=O)c5ccccc5C6=O)c(C)c4)O[Si](C)(C)C)cc3C)c3c2C(=O)c2ccccc2C3=O)c(C)c1 ; Cc1cc(C)c(Nc2ccc(Nc3c(C)cc(OCCC[Si](C)(C)O[Si](C)(C)O[Si](C)(C)CCCOc4cc(C)c(Nc5ccc(Nc6c(C)cc(C)cc6C)c6c5C(=O)c5ccccc5C6=O)c(C)c4)cc3C)c3c2C(=O)c2ccccc2C3=O)c(C)c1]

Synthesis of Dye 2 and Dye 3

a. Preparation of Mono-Allyl Modified Anthraquinone (MAA)

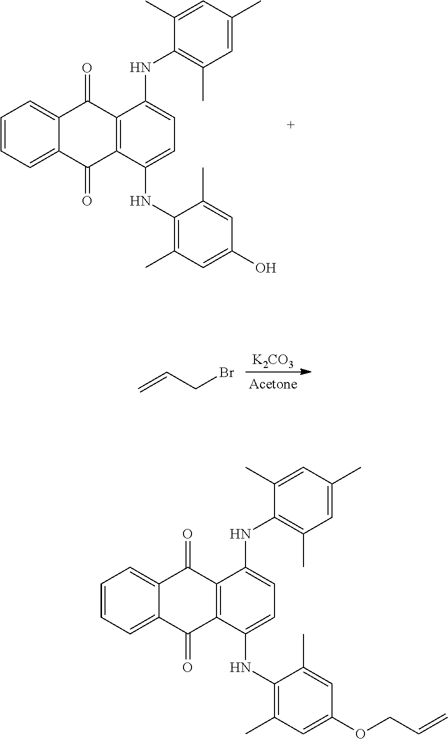

1-((4-hydroxy-2,6-dimethylphenyl)amino)-4-(mesitylamino)-anthraquinone was prepared by Example 1. A mixture of 1-((4-hydroxy-2,6-dimethylphenyl)amino)-4-(mesitylamino)-anthraquinone (400 mg, 0.83 mmol), allylbromide (1.2 equiv., 122 mg) and anhydrous K₂CO₃ (1.5 equiv., 175 mg) in dry acetone (10 mL) was refluxed under nitrogen overnight. The reaction mixture then cooled to room temperature, filtered through sintered funnel to remove solid residue and the filtrate was evaporated to dryness. The residue was purified by column chromatography using methylene chloride as eluent. The mono-allyl modified anthraquinone was obtained with the yield of 91%. ¹H NMR (CDCl₃, ppm): 11.79 (s, 1H), 11.71 (s, 1H), 8.43 (m, 2H), 7.76 (m, 2H), 6.93 (s, 2H), 6.69 (s, 2H), 6.56 (s, 2H), 6.05 (m, 1H), 5.35 (m, 2H), 4.52 (d, 2H), 2.17 (s, 3H), 2.15 (s, 12H). ESI-MS (m/z, Ion, Formula): 517.2496, (M+H)⁺, C₃₄H₃₃N₂O₃, (theoretical mass 516.24)

b. Procedure for the Preparation of Anthraquinone Dimer with Siloxane Bridge

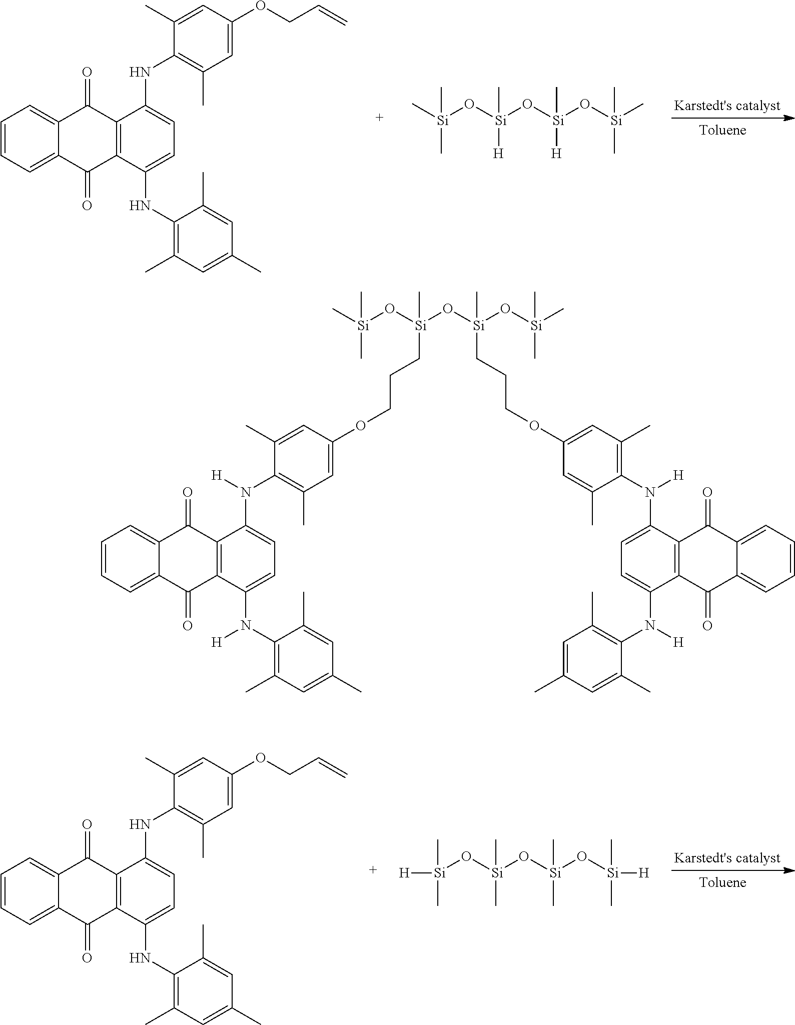

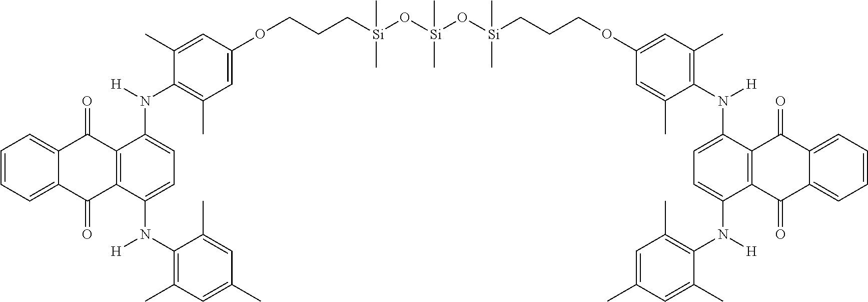 [SMILES: Cc1cc(C)c(Nc2ccc(Nc3c(C)cc(OCCC[Si](C)(C)O[Si](C)(C)O[Si](C)(C)CCCOc4cc(C)c(Nc5ccc(Nc6c(C)cc(C)cc6C)c6c5C(=O)c5ccccc5C6=O)c(C)c4)cc3C)c3c2C(=O)c2ccccc2C3=O)c(C)c1]

MAA (2.20 equiv.) was dissolved in anhydrous toluene under N₂. Siloxane (1.00 equiv.) (including 1,1,1,3,5,7,7,7-Octamethyltetrasiloxane and 1,1,3,3,5,5-Hexamethyltrisiloxane) was injected through a septum, followed by the addition of Karstedt's catalyst (platinum divinyltetramethy-siloxane complex in xylene, 3 wt). The resulting mixture was stirred at 70° C. overnight. The solution was evaporated under reduced pressure. The crude product was purified by chromatography on silica.

Properties of Dye 2

The weight loss of Dye2 at 230° C. for 1 h in air from TGA is 6.79%. Absorption spectra in PGMEA: maximum peaks at 583 nm (log ε=4.31) and 627 nm (log ε=4.33). ¹H NMR (CDCl₃, ppm): 11.68 (s, 4H), 11.60 (s, 4H), 8.31 (m, 4H), 7.64 (m, 4H), 6.81 (s, 4H), 6.54 (s, 4H), 6.44 (s, 4H), 3.78 (t, 4H), 2.17 (s, 6H), 2.03 (s, 24H), 1.70 (m, 4H), 0.51 (m, 4H), 0.01 (m, 24H). ESI-MS (m/z, Ion, Formula): 1315.5891, (M+H)⁺, C₇₆H₉₁N₄O₉Si₄, (theoretical mass 1314.58).

Properties of Dye 3

The weight loss of Dye2 at 230° C. for 1 h in air from TGA is 2.49%. Absorption spectra in PGMEA: maximum peaks at 583 nm (log E=4.45) and 627 nm (log E=4.49). ¹H NMR (CDCl₃, ppm): 11.69 (s, 4H), 11.60 (s, 4H), 8.31 (m, 4H), 7.64 (m, 4H), 6.81 (s, 4H), 6.54 (s, 4H), 6.44 (s, 4H), 3.78 (t, 4H), 2.18 (s, 6H), 2.03 (s, 24H), 1.69 (m, 4H), 0.54 (m, 4H), 0.01 (s, 12H), −0.12 (s, 6H). ESI-MS (m/z, Ion, Formula): 1241.5725, (M+H)⁺, C₇₄H₈₅N₄O₈Si₃, (theoretical mass 1240.56)

Same procedure as of Example 2 was conducted excepting for Dye2 or Dye3 were used instead of Dye 1.

### Examples 5 to 7

### Comparative Examples

Same procedure as of Example 2 was conducted excepting for the dyes disclosed below were used instead of Dye 1.

Dye Used in Example 5

1,4-bis((isopropylamino)anthraquinone (Solvent Blue 36)

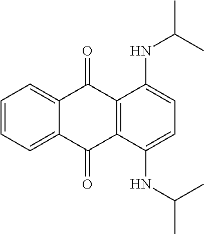 [SMILES: CC(C)Nc1ccc(NC(C)C)c2c1C(=O)c1ccccc1C2=O]

Dye Used in Example 6

1,4-bis(mesitylamino)anthraquinone (Solvent Blue 104)

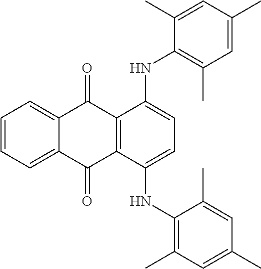 [SMILES: Cc1cc(C)c(Nc2ccc(Nc3c(C)cc(C)cc3C)c3c2C(=O)c2ccccc2C3=O)c(C)c1]

Dye Used in Example 7

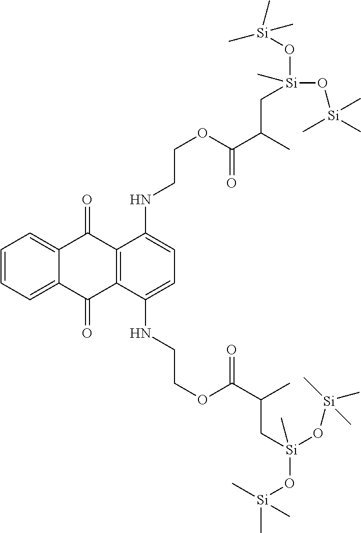 [SMILES: CC(C[Si](C)(O[Si](C)(C)C)O[Si](C)(C)C)C(=O)OCCNc1ccc(NCCOC(=O)C(C)C[Si](C)(O[Si](C)(C)C)O[Si](C)(C)C)c2c1C(=O)c1ccccc1C2=O]

(Bis-siloxane modified 1,4-dialkylamino-anthraquinone (QS) alkylamine anthraquinone)

TABLE 1

| | | Solubility in | ΔEab after baking at |
|---|---|---|---|
| Examples | Dyes | PGMEA (wt %) | 230° C. for 1 h |
| 2 | Dye 1 | 17 | 3.2 |
| 3 | Dye 2 | 11.0 | 3.6 |
| 4 | Dye 3 | 9.7 | 1.5 |
| 5 | Solvent Blue 36 | 1.92 | 33 (color almost |
| | | | disappeared) |
| 6 | Solvent Blue 104 | 0.6 | 7.0 |
| 7 | (QS) | ~4.6 | 24.5 (color changed |
| | | | from blue to violet) |

Referring to Table 1, it can be found that Examples 2 to 4 show significant improvement in both thermal stability and solubility in PGMEA compare with Examples 5 to 7.

### Example 8

### Synthesis of a First Intermediate

Another method for synthesis of a first intermediate for an asymmetric 1,4-diamino anthraquinone compound is disclosed.

A mixture of 22.1 g (91.2 mmol) of 2,3-dihydro-9,10-dihydroxy-1,4-anthracenedione, 21.9 g (91.2 mmol) quinizarin, 7.55 g (122.2 mmol) of boric acid, 250 mL n-butanol was refluxed under N₂ for 10 min. Then, 25.01 g (182.3 mmol) of 2,6-dimethyl-4-hydroxylaniline was dissolved into 100 mL NMP, sparged with N₂ and added dropwise during 2 hours. The reaction mixture was stirred at reflux under N₂ overnight. After cooling to 80° C., n-butanol was distilled off by vacuum distillation. The resulting NMP solution was reacted in air for 1 h at 80° C. The reaction was cooled to room temperature, mixed with diatomaceous earth, and extracted with acetone. The product was precipitated with water, washed with a hot 3:1 water:methanol, and dried under vacuum yielding 40.66 g product (62% yield).

### Example 9

### Synthesis of an Asymmetric Anthraquinone Compound

Another method for synthesis of an asymmetric 1,4-diamino anthraquinone compound is disclosed.

A mixture of 2.00 g of 1-((4-hydroxy-2,6-dimethylphenyl)amino)-4-hydroxyl-anthraquinone, 3.76 g of trimethylaniline, 0.40 g of boric acid, 0.40 g of zinc dust, and 4.00 g pivalic acid was heated at 160° C. for 6 hours in an oil bath under N₂. The reaction mixture was poured into 200 mL of crushed ice containing 20 mL of concentrated hydrochloric acid. The stirred mixture was then filtered to give the mixed product. Then washed with 5% hydrochloric acid, and water thoroughly. After dried, the final product was purified by silica gel column using methylene chloride and methanol as eluent. The asymmetrically substituted 1,4-diarylamino-anthraquinone derivative with blue color was obtained with the yield of 82%.

What is claimed is:

1. An anthraquinone compound having siloxane structure, represented by the following formula (1) or formula (2): 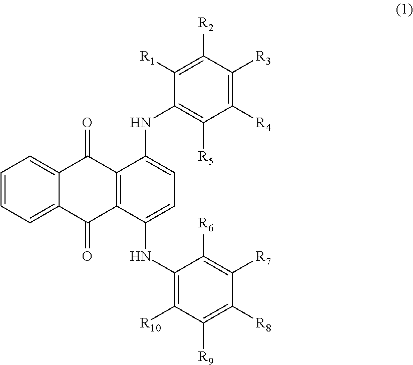
wherein R₁₋₁₀ are independently selected from the group consisting of alkyl group having 1 to 20 carbon atoms, halogen atom, hydrogen atom, hydroxyl group, cyano group, sulfonyl group, sulfo group, sulfato group, aryl group, nitro group, carboxyl group, alkoxy group having 1 to 20 carbon atoms and *—X-L-S1; wherein X is selected from the group consisting of nitrogen atom, oxygen atom, sulfur atom, sulfonyl group, sulfo group and carboxyl group; L is selected from divalent groups consisting of alkylene, oxyalkylene, cycloalkylene, oxygen atom and hetero-containing alkylene; Si is siloxane containing group represented by —(O—Si(R₁₁)(R₁₂)—)ₙ—O—Si(CH₃)₃, n is an integer from 0 to 100 and R₁₁, R₁₂ are independently selected from the group consisting of hydrogen atom, alkyl group having 1 to 20 carbon atoms and —(O—Si)ₘ—O—Si(CH₃)₃, m is an integer from 0 to 100; * means the position which bonds to the benzene ring of formula (1); and at least one of R₁₋₁₀ is *—X-L-S1: 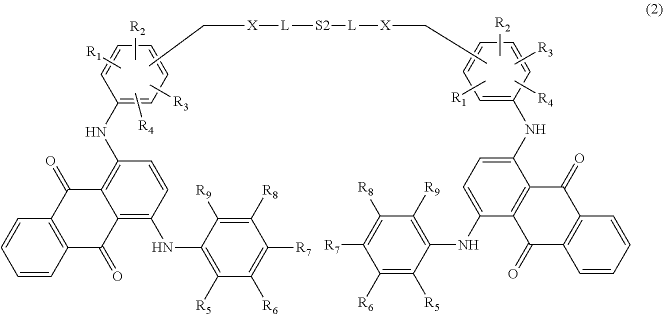
wherein R₁₋₉ are independently selected from the group consisting of alkyl group having 1 to 20 carbon atoms, halogen atom, hydrogen atom, hydroxyl group, cyano group, sulfonyl group, sulfo group, sulfato group, aryl group, nitro group, carboxyl group and alkoxy group having 1 to 20 carbon atoms, X is selected from the group consisting of nitrogen atom, oxygen atom, sulfur atom, sulfonyl group, sulfo group and carboxyl group, L is selected from divalent groups consisting of alkylene, oxyalkylene, cycloalkylene, oxygen atom and hetero-containing alkylene; S2 is siloxane containing divalent group represented by —Si(R₁₀)(R₁₁)—(O—Si(R₁₂)(R₁₃))n-O—Si(R₁₄)(R₁₅)—, R₁₀₋₁₅ are selected from the group consisting of hydrogen atom, alkyl group having 1 to 20 carbon atoms and —(O—Si)ₘ—O—Si(CH₃)₃, m is an integer from 0 to 100, n is an integer from 0 to 100.

2. The compound of claim 1, wherein at least two of R₁₋₁₀ in formula (1) are *—X-L-S1.

3. The compound of claim 1 or 2, wherein X is oxygen atom.

4. The compound of any of claims 1 to 3, wherein L is alkylene group having 1 to 3 carbon atoms.

5. The compound of any of claims 1 to 4, wherein n is 2 or less and m is 2 or less.

6. A composition comprising a resin and the compound of any of claims 1 to 5.

7. The composition of claim 6, further comprises a radiation-sensitive compound.

8. An article having a polymer layer formed from the composition of claim 6 or 7.

9. The article of claim 8, wherein the polymer layer is a negative-type layer.

10. A color filter comprising at least one the compound of any of claims 1 to 5.

11. A method for synthesis of an asymmetric 1,4-diaminoanthraquinone compound wherein the amino groups are substituted with different groups selected from alkyl, aryl and alkylaryl groups, comprising the steps of:
(A) reacting in the presence of at least one catalyst a mixture of 2,3-dihydro-9,10-dihydroxy-1,4-anthraquinone and 1,4-dihydroxyanthraquinone with a compound represented by the formula (3),
R¹—NH₂ (3)
wherein R¹ is an aryl group, alkyl group or an arylalkyl group, R¹ can be substituted by the group selected from the group consisting of hydroxyl group, amino group, thiol group, alkyl group having 1 to 20 carbon atoms, aryl group or combination thereof to form a first intermediate, and
(B) reacting in the presence of at least one catalyst the first intermediate with a compound represented by the formula (4)
R²—NH₂ (4)
wherein R² is an aryl group, alkyl group or an arylalkyl group, R² can be substituted by the group selected from the group consisting of hydroxyl group, amino group, thiol group, alkyl group having 1 to 20 carbon atoms, aryl group or combination thereof, and R¹ and R² are different.

12. The method of claim 11 wherein R¹ of the formula (3) is aryl group and R² of the formula (4) is aryl group, R¹ and R² are substituted by the group selected from the group consisting of hydroxyl group, amino group, thiol group, alkyl group having 1 to 20 carbon atoms, aryl group or combination thereof, and the substituent of R¹ and the substituent of R² are different.

13. A method for synthesis of an asymmetric 1,4 bis(arylamine)anthraquinone compound, comprising the steps of:
(A) reacting in the presence of boric acid a mixture of 2,3-dihydro-9,10-dihydroxy-1,4-anthraquinone and 1,4-dihydroxyanthraquinone with a compound represented by the formula (5)
Ar¹—NH₂ (5)
wherein Ar¹ is aryl group substituted by at least one group selected from the group consisting of hydroxyl group, amino group, thiol group, alkyl group having 1 to 20 carbon atoms, aryl group or combination thereof to form a first intermediate, and
(B) reacting in the presence of boric acid, metal zinc and an acid the first intermediate with a compound represented by the formula (6)
Ar²—NH₂ (6)
wherein Ar² is aryl group substituted by at least one group selected from the group consisting of hydroxyl group, amino group, thiol group, alkyl group having 1 to 20 carbon atoms, aryl group or combination thereof, and the substituent of Ar¹ and the substituent of Ar^{e} are different.

14. The method for claim 13, wherein the mole ratio of 2,3-dihydro-9,10-dihydroxy-1,4-anthraquinone and 1,4-dihydroxyanthraquinone is 9:1 to 1:1.

15. The method for claim 13 or 14, wherein the acid is pivalic acid.
